(19) United States
(12) Patent Application Publication
(10) Pub. No.: US 2025/0082404 A1
(43) Pub. Date: Mar. 13, 2025

(54) LASER ALIGNMENT SYSTEM

(71) Applicant: Alcon Inc., Fribourg (CH)

(72) Inventors: Mark Harrison Farley, Laguna Hills, CA (US); Ronald Dean Smith, Parker, CO (US)

(21) Appl. No.: 18/825,152

(22) Filed: Sep. 5, 2024

Related U.S. Application Data
(60) Provisional application No. 63/582,011, filed on Sep. 12, 2023.

(51) Int. Cl. A61B 18/20

(52) U.S. Cl. CPC A61B 18/20; A61B 2018/2015; A61B 2018/20553

(57) ABSTRACT

The present disclosure generally relates to systems and methods for laser alignment, and more particularly, systems and methods for alignment of an optical element for laser transmission. In certain embodiments, a cover plate is configured to slide into place on an optic mounting plate. Positioning the cover plate applies force to a biasing element to press an optical element against a registration surface of the optic mounting plate to align the optical element with an optical aperture. The cover plate may be secured relative to the optic mounting plate to retain the biasing element and the optical element.

### BACKGROUND

In a wide variety of medical procedures, laser light is used to assist the procedure and treat patient anatomy. For example, in laser photocoagulation performed during retinal detachment surgery, a laser probe is used to cauterize blood vessels at laser burn spots across the retina.

Certain types of laser probes burn multiple spots at a time, which may result in faster and more efficient photocoagulation. For example, a laser probe may be coupled, through an optical fiber cable, to a surgical laser system that splits a single laser beam into multiple laser beams that exhibit a laser spot pattern and delivers the laser beams to an array of individual optical fibers (“fibers”) in the optical fiber cable that exhibit a corresponding fiber pattern. At their distal ends, the fibers are coupled to the laser probe and project the laser beam spots with the laser spot pattern onto the retina. Typically, the fibers are tightly packed together so that the fiber pattern matches the laser spot pattern. The laser spot pattern may be created by passing the laser light through a diffractive element to split the laser into the spot pattern and align the laser spot pattern with the corresponding fibers.

Generally, the diffractive element is precisely positioned and angularly oriented to properly form a multi-spot laser spot pattern and accurately direct the laser spot pattern to the corresponding cores or fibers of the optical fiber cable. A compounding aspect of positioning the diffractive element includes the use of an aiming laser in tandem with the treatment laser. In such a system, just as the treatment laser, the aiming laser is passed through a diffractive element to align with the fibers of the optical fiber cable. Maintaining alignment of both diffractive elements is therefore both sensitive and critical.

### SUMMARY

According to certain embodiments, the present disclosure is directed to a laser alignment system. In certain embodiments, the laser alignment system includes an optic mounting plate, a biasing element, and a cover plate. The optic mounting plate includes an optic mounting receptacle, an optical aperture, and a biasing recess. The optic mounting receptacle is formed on a first side of the optic mounting plate. The optic mounting receptacle is configured to accept an optical element. The optic mounting receptacle includes a registration surface formed on a periphery of the mounting receptacle. The registration surface faces inward on the optic mounting receptacle. The optical aperture is disposed within the optic mounting receptacle. The optical aperture extends through a thickness of the optic mounting plate from the first side of the optic mounting plate to a second side of the optic mounting plate. The biasing recess is formed in the optic mounting plate to extend along two sides of the optic mounting receptacle. The biasing element is disposed in the biasing recess and shaped to extend along the two sides of the optic mounting receptacle to apply a biasing force to the optical element to press the optical element against the registration surface to align the optical element relative to the optic mounting plate. The cover plate is configured to couple to the first side of the optic mounting plate to cover the biasing recess. The cover plate is configured to engage the biasing element with a contact surface of the cover plate and to load the biasing element to apply the biasing force to the optical element.

### BRIEF DESCRIPTION OF THE DRAWINGS

For a more complete understanding of the present technology, its features, and its advantages, reference is made to the following description, taken in conjunction with the accompanying drawings, in which:

FIG. 1 illustrates a plan view of a system for generating laser beams for delivery to a surgical target, in accordance with certain embodiments of the present disclosure.

FIG. 2 illustrates a plan view of an example of a surgical laser system, and the components therein, in accordance with certain embodiments of the present disclosure.

FIG. 3 illustrates a perspective view of an optic mounting system that may be used in conjunction with a surgical laser system, such as the surgical laser system of FIG. 2, in accordance with certain embodiments of the present disclosure.

FIG. 4 illustrates a perspective view of an optic mounting plate of the optic mounting system of FIG. 3, in accordance with certain embodiments of the present disclosure.

FIG. 5 illustrates an assembly view of the optic mounting system of FIG. 3, in accordance with certain embodiments of the present disclosure.

FIG. 6 illustrates a method of using the optic mounting system of FIG. 3, in accordance with certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body is in proximity to, for example, a surgical laser system.

As used herein, the term “about” may refer to a +/−10% variation from the nominal value. It is to be understood that such a variation can be included in any value provided herein.

Particular embodiments disclosed herein provide methods and systems for aligning multi-core fibers with multi-spot laser beam patterns of laser surgical systems.

Some ophthalmic surgical laser systems generate both an aiming beam and a treatment beam, which allows a surgeon to observe and position the aiming beam before delivering a pulse of the treatment beam to treat a target surgical site. Such surgical laser systems may also provide diffracted multi-beam pattern delivery, in which both the aiming beam and treatment beam are diffracted into identical patterns that are subsequently combined onto the same optical path for delivery to the patient through a multi-core optical fiber, a focusing objective, or a combination of these or other means.

To align aiming beams and treatment beams, separate diffractive optical elements (DOEs) for the aiming beams and treatment beams may be used. However, when separate DOEs are used, a precise clocking orientation for each of the different DOEs, with precise angular registration thereof and identical diffraction angles for aiming and treatment beam wavelengths is instrumental. The present disclosure provides systems and methods for precise alignment of both aiming and treatment beams using two separate DOEs.

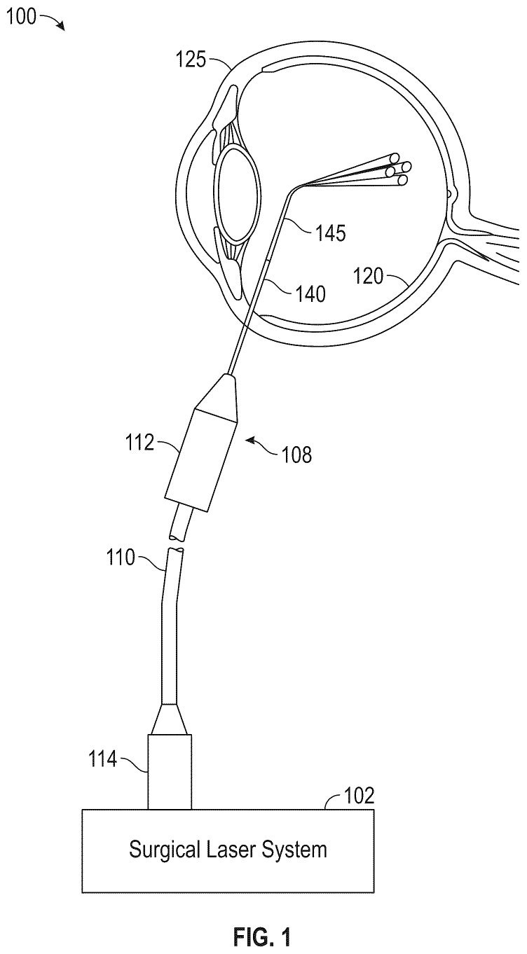

FIG. 1 illustrates an example system 100 for performing a laser-assisted ophthalmic procedure. The system 100 includes a surgical laser system 102 having one or more laser sources for generating laser beams. For example, a first laser source within surgical laser system 102 may generate a treatment beam with a first wavelength (e.g., ˜532 nanometers (nm)) while a second laser source may generate an aiming beam with a second wavelength (e.g., ˜639 nm). A user, such as a surgeon, may first trigger the surgical laser system 102 (e.g., via a foot switch, voice commands, etc.) to emit the aiming beam onto a desired retinal spot. Once the surgeon has positioned the laser probe so as to illuminate the desired retinal spot with the aiming beam, the surgeon activates the treatment beam, such as through a foot pedal or other means, to treat patient anatomy (e.g., photocoagulate the desired retinal spot using the treatment beam).

As shown, the surgical laser system 102 includes a connector or port adapter 114 that couples to an optical port of the surgical laser system 102. FIG. 1 also shows an optical fiber 110 inside an optical fiber cable 111 having a distal end that couples to and extends through a probe 108 and a proximal end that couples to and extends through port adapter 114. In some cases, as further described herein, the optical fiber 110 may include more than one fiber. In the example of FIG. 1, the port adapter 114 includes a ferrule with an opening in which the proximal end of the optical fiber 110 is inserted. The proximal end of the optical fiber 110 includes an interface plane (also referred to as a proximal entrance plane) upon which laser beams from the surgical laser system 102 may be focused when the proximal end of the optical fiber 110 is inserted into the ferrule. The interface plane of the optical fiber 110 comprises the exposed proximal ends of the one or more cores where laser beams may be directed. In the example of FIG. 1, the optical fiber 110 is a multi-core optical fiber (MCF) with four cores. As such, the interface plane of the proximal end of the optical fiber 110 comprises the proximal ends of the four cores upon which laser beams may be focused.

The surgical laser system 102 may be configured to split a single laser beam that is generated by a laser source into multiple laser beams that exhibit a laser spot pattern. For example, the surgical laser system 102 may split an aiming beam into four aiming beams and then deliver the four aiming beams to the interface plane of the optical fiber 110 through the opening of the ferrule of the port adapter 114. The surgical laser system 102 may further be configured to split the treatment beam into four treatment beams and deliver the four treatment beams to the interface plane of the optical fiber 110 through the opening of the ferrule. In such an example, each of the cores of the optical fiber 110 would then be transmitting a multi-wavelength or combined beam, which may refer to a treatment beam combined with an aiming beam. Though certain aspects are described with respect to the cores of the optical fiber transmitting a combined beam, it should be noted that the cores of the optical fiber 110 can also individually transmit either the treatment beam or the aiming beam, depending on which beam(s) are activated and incident on the optical fiber 110.

In some examples, the surgical laser system 102 may also propagate an illumination beam into an interface plane of the optical fiber 110 (e.g., which may also include a proximal end of a cladding that holds the cores within the optical fiber 110) in order to illuminate the inside of the eye, especially areas of the retina 120 that are to be photocoagulated. In certain aspects, an illumination beam may be generated by a white light-emitting diode (LED).

The optical fiber 110 delivers the combined beams to the probe 108, which propagates a multi-spot pattern (e.g., four spots) of combined beams to the retina 120 of a patient's eye 125. The probe 108 includes a probe body 112 and a probe tip 140 that house and protect the distal end of the optical fiber 110. A distal end portion 145 of the probe tip 140 may also contain a lens that focuses the combined beams on the retina 120.

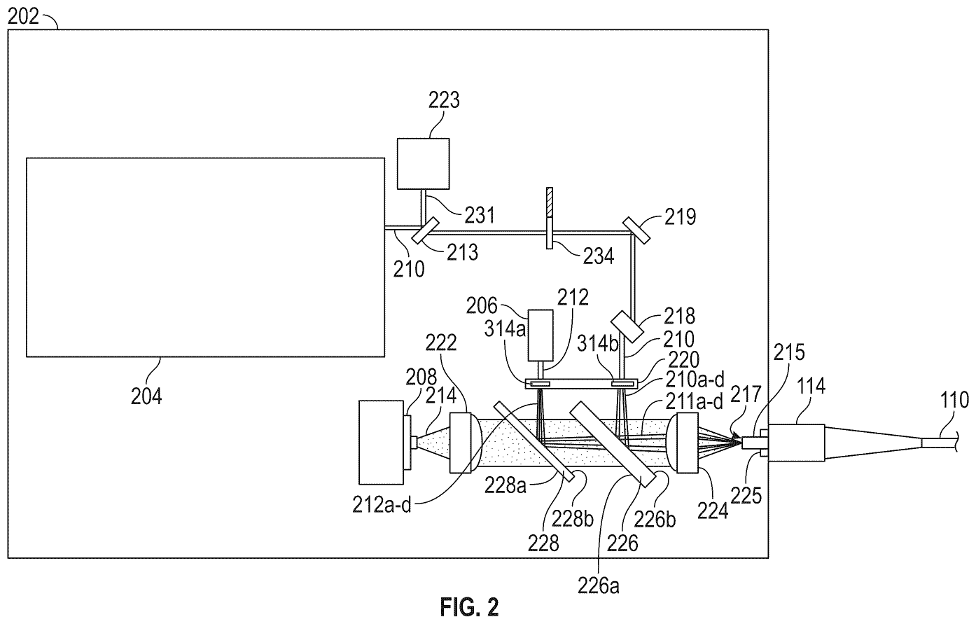

FIG. 2 illustrates an example of a surgical laser system 202, and the components therein, that may be implemented according to embodiments described herein. The surgical laser system 202 comprises a laser source 204, which propagates a treatment beam 210, a laser source 206, which propagates an aiming beam 212, and a light source 208, which propagates an illumination beam 214. The surgical laser system 202 further includes a plurality of lenses, diffractive elements, beam splitters, and other optical relay devices for relaying the laser and illumination beams between their respective sources and desired ports, which may together be referred to as an “optical relay system.”

At the outset of the surgery, a surgeon may activate the light source 208 in order to illuminate the inside of the eye's globe and make it easier to view the retina. As shown, once emitted by the light source 208, the illumination beam 214 (stippled segment) is received by a collimating lens 222, which is configured to produce a beam with parallel rays of light. In certain embodiments, the collimating lens 222 may be a multi-element achromat comprising two singlet lenses and one doublet lens. Therefore, as shown, the illumination beam 214 emerges with parallel rays of light from the other side of the collimating lens 222 and passes through beam splitters 228 and 226 (which may also be referred to as dichroic mirrors), respectively, to reach a condensing lens 224.

In certain embodiments, the condensing lens 224 may be a multi-element achromat comprising two singlet lenses and one doublet lens. In such embodiments, the condensing lens 224 has the same design as the collimating lens 222, except that the assembly is reversed (e.g., rotated by 180 degrees), thereby creating a one-to-one magnification imaging system. Each of the beam splitters 228 and 226 may have different coatings on their two sides, 228a and 228b, and 226a and 226b, respectively. For example, the sides 228a and 226a are coated such that they allow light propagated thereon to pass through the beam splitters 228 and 226. As such, the illumination beam 214, which is propagated onto the sides 228a and 226a, passes through those surfaces of the beam splitters 228 and 226. On the other hand, the sides 228b and 226b are coated to reflect light or laser beams such as the aiming beam 212 and the treatment beam 210, respectively, as further described below.

The condensing lens 224 then converges the illumination beam 214 into an interface plane of a proximal end of an optical fiber, such as the optical fiber 110, which is coupled to the port 225 of the surgical laser system 202 through the port adapter 114. As described in relation to FIG. 1, the optical fiber 110 may have four cores embedded within a larger-diameter cladding. As such, the condensing lens 224 focuses the illumination beam 214 into an interface plane of the optical fiber 110 such that the illumination beam 214 is propagated, along an entire length of the cladding and each of the four cores of the optical fiber 110, to the distal end of a surgical probe (e.g., the probe 108 of FIG. 1) that is coupled to the optical fiber 110. As described above, the interface plane of the optical fiber 110 comprises the proximal ends of the four cores and cladding thereof that are exposed through the opening 217 of the port adapter 114, respectively, via the ferrule 215.

Once the surgeon is able to view inside the eye's globe, the surgeon may project, from the distal end of the probe, one or more desired aiming beam spots onto the retina. More specifically, after activation by the surgeon, the laser source 206 emits the aiming beam 212 (e.g., a red laser beam) onto/through an optical element 314 (e.g., a diffractive optical element (DOE)) of an optic mounting system 220. In some embodiments the optical element 314 diffracts the aiming beam 212 into a desired number of aiming beams based on the region of the optical element 314 (hereinafter, referred to as a “DOE region” or “region”) through which the aiming beam 212 passes. Generally, the optic mounting system 220 may have two or more optical elements 314 coupled thereto, and each optical element 314 may have one or more regions for forming different numbers of beams and/or shapes from aiming beam 212.

In the example of FIG. 2, the optic mounting system 220 is positioned such that aiming beam 212 is aligned with one of two optical elements 314a-b coupled to the optic mounting system 220, and more specifically, a region of the optical element 314a that diffracts the aiming beam 212 into the aiming beams 212a-d (e.g., four aiming beams). However, a surgeon may change the position of optic mounting system 220, and thus the optical element 314a-b, in order to diffract a beam into a different number of beams (e.g., 2 or 1). For example, using voice command or some other feature of the surgical laser system 202, a surgeon may position the optic mounting system 220 and thereby, the optical element 314a, to align the aiming beam 212 with a different region of the optical element 314a, which may diffract the aiming beam 212 into two, or one, or other numbers of beams.

Once diffracted, the resulting aiming beams may be reflected by the beam splitter 228, through the beam splitter 226, and onto the condensing lens 224. In examples where the aiming beams 212a-d are red aiming beams, the beam splitter 228 may be a red dichroic optical element, and the aiming beams 212a-d may reflect off of a narrowband red spectral notch in the beam splitter 228. The condensing lens 224 may then focus the four aiming beams onto the interface plane of a proximal end of the optical fiber 110 such that each of the aiming beams is propagated, along an entire length of a corresponding core of the optical fiber 110, to the distal end of a surgical probe (e.g., the probe 108 of FIG. 1). Each of the four aiming beams focuses with high coupling efficiency into the corresponding core within the 4-core MCF, and propagates down the length of the core to the distal end of the MCF. This allows the surgeon to project from the distal end of the probe four desired aiming beam spots onto the retina.

In some embodiments, the light source 208 propagates a white light illumination beam 214 that passes through the side 228a of the beam splitter 228, which may have an anti-reflective coating, and is incident on the side 228b of the beam splitter 228, which may have a red dichroic coating. In such embodiments, a portion of the white light illumination beam 214 within a red-reflecting spectral region of the red dichroic coating on the side 228b is strongly reflected by the red dichroic coating, while a remainder of the white light illumination beam 214 will substantially transmit through the coating.

In some embodiments, the white light illumination beam 214 further passes through the beam splitter 226, which may have corresponding coatings on one or both sides to filter light incident on the beam splitter 226. For example, the beam splitter 226 may include an anti-reflective coating on the side 226a and a green dichroic coating on the side 226b. A portion of the white light illumination beam 214 within a narrowband green-reflecting spectral region of the green dichroic coating on the side 226b will thus be strongly reflected by the green dichroic coating, while the remainder of the white light illumination beam 214 will substantially transmit through the green dichroic coating. Although red and green spectral portions of the white spectrum of illumination beam 214 may be strongly reflected by coatings on sides 228b and 226b, respectively, the transmitted portion of the white light illumination beam 214 reaching the lens 224 may remain substantially white in color.

As described above, once the surgeon has positioned and activated the laser probe so as to project aiming beam spots onto the retina, the surgeon activates the laser source 204, such as through a foot pedal or other means, to treat patient anatomy (e.g., photocoagulate the desired retinal spot using the treatment beam). When activated, the laser source 204 emits the treatment beam 210, e.g., a green laser beam, as shown in FIG. 2. The treatment beam 210 reaches the beam splitter 213, which is configured to allow a substantial portion of the treatment beam 210 to pass through, while reflecting a trivial portion 231 onto a sensor 223. The sensor 223 is a light sensor configured to detect whether the laser source 204 is active or not and to monitor a power level of the treatment beam. After passing through the beam splitter 213 and provided that the shutter 234 is in an open position to permit the treatment beam 210, the treatment beam 210 is received at a fixed fold mirror 219, which is configured to reflect the treatment beam 210 onto the beam splitter 218.

In certain embodiments, the surgical laser system 202 may also include a shutter 234 arranged between the laser source 204 and the fixed fold mirror 219. The shutter 234 may be configured to alternatively block or permit the treatment laser beam 210 from reaching the fixed fold mirror 219. A surgeon or surgical staff member can control the shutter 234 (e.g., via a foot switch, voice commands, etc.) to emit the laser aiming beam and fire the treatment laser beam (i.e., open the shutter 234) to treat patient anatomy (e.g., photocoagulation). In each case, the beam splitter 218 may direct the laser beams towards the port adapter 114.

As shown, the treatment beam 210 passes through beam splitter 218 before reaching a second optical element 314b coupled to the optic mounting system 220. In FIG. 2, the second optical element 314b of the optic mounting system 220 is shown as diffracting the treatment beam 210 into treatment beams 210a-210d (e.g., four treatment beams). However, a surgeon may change the position of the optic mounting system 220, and thus the second optical element 314b, in order to align the treatment beam 210 with a different region of the second optical element 314b and diffract the beam 210 into a different number of beams (e.g., 2 or 1). For example, using voice command or some other feature of surgical laser system 202, a surgeon may position the optic mounting system 220 to align the treatment beam 210 with a different region of the second optical element 314b, which may diffract the treatment beam 210 into two, or one, or other numbers of beams.

The treatment beams 210a-210d are then received at the beam splitter 226, which reflects the treatment beams 210a-210d onto the condensing lens 224. In examples where the treatment beams 210a-d are green treatment beams, the beam splitter 226 may be a green dichroic optical element, and the treatment beams 210a-d may reflect off a narrowband green spectral notch in the beam splitter 226. The treatment beams 210a-d are reflected by the beam splitter 226 at an angle with respect to the beam splitter 226 that is equal to the angle with which the aiming beams 212a-d are passed through the beam splitter 226. Therefore, when the laser source 204 is active, the transmitted treatment beams 210a-d and the aiming beams 212a-d are combined (e.g., such that they overlay each other) creating combined beams 211a-d), before reaching the condensing lens 224.

The condensing lens 224 focuses the combined beams 211a-211d onto an interface plane of the proximal end of the optical fiber 110 such that each of the combined beams 211a-211d is propagated, along an entire length of a corresponding core of the optical fiber 110, to the distal end of a surgical probe (e.g., the probe 108 of FIG. 1). More specifically, in the example of FIG. 2, the optical fiber 110 is an MCF with four cores, such as cores A, B, C, and D. In such an example, the condensing lens 224 focuses the combined beams 211a-211d onto an interface plane of a proximal end of the optical fiber 110 such that, for example, combined beam 211a is propagated onto core A, the combined beam 211b is propagated onto core B, the combined beam 211c is propagated onto core C, and the combined beam 211d is propagated onto core D.

Generally, embodiments of the present disclosure provide several advantages. For example, certain embodiments described herein provide consistent positioning and angular orientation of one or more DOEs within an optic mounting system by pressing the DOEs against a precision registration surface with a biasing element. Further, certain embodiments described herein provide a simple assembly by incorporating a cover plate configured to at least partially enclose the DOEs and the biasing element while engaging the biasing element to apply the biasing force on the DOEs. Installation of the cover plate provides a consistent engagement of the biasing element to allow for repeatable and predictable self-alignment of the DOEs with ease and simplicity of assembly. Embodiments described herein may also be scalable and provide for more compact size, for example, along an optical path. Certain embodiments described herein may also be compatible with translation and rotational movement with high stability at relatively high rates of movement. As described in more detail below, the optical elements incorporated may be interchangeable to suit a particular function or performance characteristic.

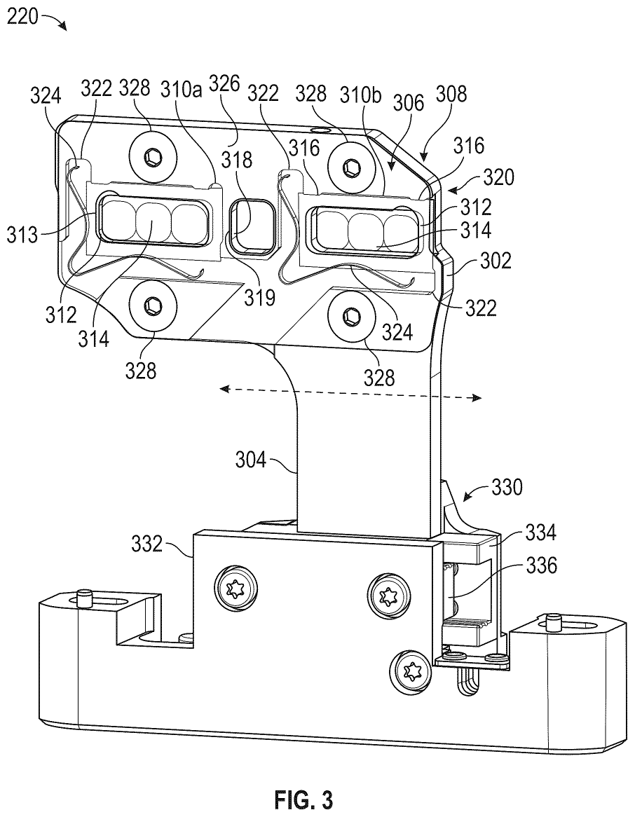

FIG. 3 illustrates a perspective view of the optic mounting system 220 in FIG. 2. Again, the optic mounting system 220 facilitates positioning and alignment of optical elements 314 (314a and 314b are shown) to produce a desired laser beam pattern and/or perform other manipulation of laser beam(s) directed through the optical elements 314, as well as for precisely directing the laser beam(s) to the optical fiber 110 of FIG. 2.

As shown, the optic mounting system 220 includes an optic mounting plate 302. In some embodiments, the optic mounting plate 302 has an L-shaped geometry with a vertical leg 304 coupled to a base plate 331. In some embodiments, the vertical leg 304 and the base plate 332 provide translational positioning of, e.g., optical elements 314. In some embodiments, the leg 304 may include other components to facilitate movement of the system 220 as described below. The optic mounting plate 302 may be substantially planar and have a first side 306 opposing a second side 308 which together define a thickness of the optic mounting plate 302. In some embodiments, the optic mounting plate 302 is formed of metallic materials (e.g., aluminum, steel, etc.) or composite materials (e.g., thermoplastic polymers). In some embodiments, the materials of the optic mounting plate 302 may provide desired thermal properties (e.g., heat dissipation), have sufficient resistance to bending induced by relatively rapid movement, and/or the like.

In some embodiments, the optic mounting plate 302 includes at least one optic mounting receptacle 310. In the illustrated embodiment, the optic mounting plate 302 is shown as having two separate optic mounting receptacles 310a and 310b. In some embodiments, each optic mounting receptacle 310 may be designated for a particular purpose (e.g., one or more receptacles 310 to pass a treatment beam and one or more receptacles 310 to pass an aiming beam). In some embodiments, the optic mounting receptacles 310 may be agnostic with respect to the beams passed through them. In other words, one or more of the optic mounting receptacles 310 may be used for either a treatment beam or an aiming beam. Other embodiments, however, may include fewer or more optic mounting receptacles 310.

Generally, the optic mounting receptacles 310 are each shaped to accept an optical element 314, which in some embodiments comprises a diffractive optical element (DOE). The optic mounting receptacles 310 may be sized to accommodate an optical element 314, such as a DOE, having multiple regions with diffractive properties or characteristics. In the illustrated embodiment, the optical elements 314 have three separate regions. In other embodiments, the optical elements 314 may have fewer or more regions. In some embodiments, each optical element 314 may have regions disposed in a linear arrangement to facilitate translational linear movement of the optic mounting plate 302 to pass laser beams through a desired one of the regions. In embodiments where a plurality of optical elements 314 are mounted in two or more optic mounting receptacles 310, each optical element 314 may have regions corresponding to regions of the other mounted optical elements 314 such that translational or rotational movement of the optic mounting plate 302 causes laser beams to pass through desired corresponding regions of each optical element 314. In other words, the plurality of optical elements 314 may have corresponding or synchronous regions.

In some embodiments, the optic mounting receptacles 310 may each include one or more registration surfaces 316. The registration surfaces 316 may be formed on a periphery of the optic mounting receptacles 310. The registration surfaces 316 may be positioned within the optic mounting receptacles 310 to align the optical elements 314 relative to the optic mounting plate 302. In certain embodiments, the registration surfaces 316 may protrude inward toward the apertures 312 and have planar surfaces. However, other geometries may also incorporated into the registration surfaces 316.

In some embodiments, the optic mounting receptacles 310 are each coupled to one or more biasing recesses 322. In some examples, as shown in FIG. 3, each optic mounting receptacle 310 is coupled to two or more biasing recesses 322 formed into the optic mounting plate 302, which accept and retain biasing elements 324 for biasing the optical elements 314 against the registration surfaces 316. Biasing the optical elements 314 against the registration surfaces 316 facilitates alignment of the optical elements 314 relative to the corresponding optical apertures 312. In some embodiments, the biasing recesses 322 may be formed in the optic mounting plate 302 to extend along one or more sides of each of the optic mounting receptacles 310. In some embodiments, the biasing recesses 322 are formed with the same depth, or thickness, in the optic mounting plate 302 as the optic mounting receptacles 310. In other embodiments, the biasing recesses 322 may have a different depth within the optic mounting plate 302 as compared to the optic mounting receptacles 310. In the illustrated embodiment, each of the biasing recesses 322 are formed along a bottom and left-most side of each of the optic mounting receptacles 310, however, in other embodiments, at least one of the biasing recesses 322 may be positioned at least partially along a top or other side of the optic mounting receptacles 310. In some embodiments, the biasing recesses 322 may have one or more geometries that are longer or shorter than the optic mounting receptacles 310. In other words, one or more of the biasing recesses 322 may extend beyond, or may not extend to an edge of, one or more of the optic mounting receptacles 310. In other embodiments, the biasing recesses 322 may match the optic mounting receptacles 310 along at least one edge.

In some embodiments, one or more biasing elements 324 are disposed in each biasing recess 322. The biasing elements 324 may each be shaped to extend along one of two sides of the optic mounting receptacles 310. The biasing elements 324 may be configured to press the optical element 314 against the registration surfaces 316 to align the optical element 314 relative to the optic mounting plate 302. In some embodiments, the biasing elements 324 comprise springs. For example, the biasing elements 324 may be flat or “blade” springs with multi-curve geometries that are flexible with elastic deformation in response to a compression force applied to the biasing element 324. When compressed, each biasing element 324 may generally form an angle to fit within the corresponding biasing recess 322. In some embodiments, the biasing elements 324 are non-symmetrical. For example, the biasing elements 324 may have vertically oriented portions that are shorter than horizontally oriented portions, or vice versa. In other embodiments, the biasing elements 324 are symmetrical. Embodiments of the optic mounting receptacles 310, registration surfaces 316, and biasing elements 324 are described in more detail below.

Embodiments of the optic mounting plate 302 may include one or more optical apertures 312. In some embodiments, the optical apertures 312 are disposed within the optic mounting receptacles 310. The optical apertures 312 extend through a thickness of the optic mounting plate 302. In other words, the optical apertures 312 form a through-hole in the optic mounting plate 302. The optical apertures 312 may be shaped to allow laser light to pass through the optic mounting plate 302. In some embodiments, each of the optical apertures 312 may have a similar geometry. In other embodiments, one or more of the optical apertures 312 may have a geometry distinct from another of the optical apertures 312.

In some embodiments, the optic mounting plate 302 includes one or more pass-throughs 318. In the illustrated embodiment, an example of the pass-through 318 is disposed between the optical apertures 312. The pass-through 318 may be formed in the optic mounting plate 302 to allow a first laser light to pass through the optic mounting system 220 without impinging on an optical element 314. For, example, the pass-through 318 may be used to allow a single laser beam to be passed into a fiber optic cable, passed to a laser power measurement device, or the like, without any diffraction or other manipulation by an optical element 314. In some embodiments, a cutout 320 may be formed in an edge of the optic mounting system 220 to allow a second laser light to pass by the optic mounting system 220 simultaneously with the laser light passing through the pass-through 318. In some embodiments, a spacing of the cutout 320 and the pass-through 318 is similar to a spacing of the optical apertures 312 to pass a corresponding one of the aiming and treatment lasers such that shifting the optic mounting system 220 results in laser light that previously passed through the optical apertures 312 may then pass through the pass-through 318 and the cutout 320 to avoid any effect from the optical element 314.

Embodiments of the optic mounting system 220 may include a cover plate 326. The cover plate 326 may be configured to couple to the first side 306 of the optic mounting plate 302 to retain the optical elements 314 and the biasing elements 324, and to apply a biasing force to align the optical elements 314 within the optic mounting system 220. In some embodiments, the cover plate 326 may be coupled to the optic mounting plate 302 via hardware coupling element 328. In other embodiments, the cover plate 326 may be coupled to the optic mounting plate 302 via adhesive, chemical, magnetic, electro-magnetic, or other coupling means. In the illustrated embodiment, the hardware coupling elements 328 include machine screws. In other embodiments, other hardware coupling elements may be used. Other examples of hardware coupling elements 328 comprise pins, latches, clips, toggles, nuts, bolts, and the like.

In some embodiments, the cover plate 326 has a geometry that matches at least a portion of the geometry of the base plate 332 of optic mounting plate 302. For example, the cover plate 326 may have a geometry that matches at least a portion of a geometry of the optic mounting plate 302. Similar to the optic mounting plate 302, the cover plate 326 may include optical apertures 313 that correspond, or align, with the optical apertures 312 and a pass-through 319 that corresponds, or aligns, with pass-through 318. Additional details relating to embodiments of the cover plate 326 are provided below.

In some embodiments, as shown in FIG. 3, the leg 304 of the optic mounting plate 302 extends vertically downward from the optic mounting plate 302. The leg 304 may be configured to couple the mounting plate 302 to a translation system 330. The translation system 330 may be configured to facilitate in-plane translation of the optic mounting system 220 as illustrated in FIG. 3. In other embodiments, other or additional hardware may be incorporated to allow for other movement or positional adjustment, such a rotational movement, of the optic mounting system 220. Movement of the optic mounting system 220 may be effected to adjust positioning of the optical element element(s) 314 relative to beam path(s) of incoming aiming beams and/or treatment beams.

In some embodiments, the translational system 330 may include a c-channel 334 configured to interface with a runner 336 such that the runner 336 translates within the c-channel 334 to allow for relative movement of the optic mounting system 220 relative to a base plate 332. The base plate 332 may be fixed while the optic mounting system 220 is coupled to a motor (e.g., a servo, stepper, solenoid, or other type of electromechanical or pneumatic motor) to translate the optic mounting system 220.

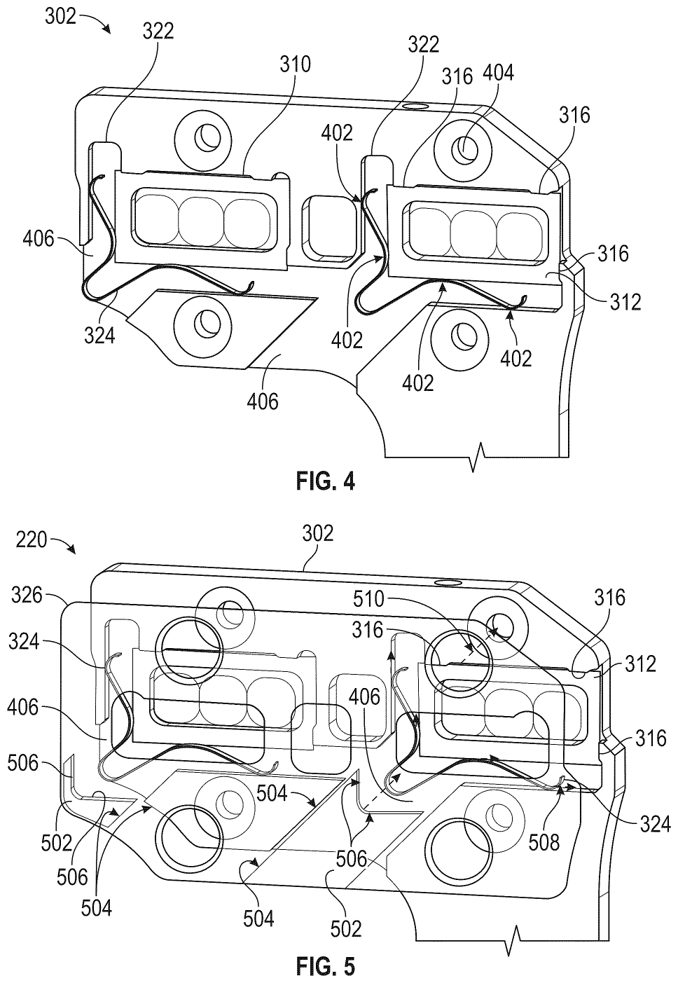

FIG. 4 illustrates an enlarged perspective view of the optic mounting plate 302. As shown, when the biasing elements 324 are situated in the biasing recesses 322, the biasing elements 324 have contact points 402 against surfaces of the biasing recesses 322 and surfaces of the optical element 314. In some embodiments, the biasing elements 324 and/or contact points 402 are slidable relative to the biasing recesses 322 and the optical element 314, e.g., against the surface of the biasing recesses 322 and surfaces of the optical element 314. In other words, some embodiments of the biasing elements 324 may not be adhered or otherwise secured to the biasing recesses 322 or the optical element 314. This allows for the biasing elements 324 to interface with the optical elements 314 with a large tolerance for initial installation of the optical elements 314 and still allows for alignment of the optical elements 314 by pressing the optical elements 314 into place against the registrations surfaces 316.

As the biasing element 324 applies a biasing force to the optical element 314, the optical element 314 is pressed against the registration surfaces 316. The registration surfaces 316 are formed within a tolerance range configured to provide precise positioning and angular orientation of the optical element 314 so as to direct laser light, such as treatment beams 210 or aiming beams 212, incident at the optical element 314 toward an optical fiber or other target in the correct pattern and with precise alignment. In some embodiments, the registration surfaces 316 extend inward into the optic mounting receptacle 310. In some embodiments, the registration surfaces 316 are flat where they contact the optical element 314. In other embodiments, the registration surface 316 may be partially cylindrical. In some embodiments, the registration surfaces 316 may be partially spherical. In still other embodiments, the registration surface 316 may come to a point where they contact the optical element 314. However, other shapes are also contemplated.

In some embodiments, the registration surfaces 316 are formed to be near one or more corners of the optical element 314, such as three corners of the optical element 314. Additional registration surfaces 316 may be positioned to be more centrally located relative to the optical element 314. In the illustrated embodiment, three registration surfaces 316 are shown for each optical element 314. However, fewer or more registration surfaces 316 may be used.

Embodiments of the optic mounting plate 302 may also include hardware mounting points 404. The hardware mounting points 404 may facilitate coupling of the cover plate 326 to the optic mounting plate 302. In the illustrated embodiment, the hardware mounting points 404 are threaded and countersunk screw holes. In other embodiments, the hardware mounting points 404 may facilitate the use of other coupling means (e.g., pins, toggles, clips, magnets, etc.).

In some embodiments, the optic mounting plate 302 includes guide channels 406. The guide channels 406 may be formed in the optic mounting plate 302 to be at a same or similar depth as at least one of the biasing recess 322 and/or the optic mounting receptacle 310. The guide channels 406 may extend from a periphery of the optic mounting plate 302 to the biasing recess 322. The guide channels 406 may be oriented at an angle relative to an edge of the optic mounting plate 302 and/or the optical elements 314. The guide channels 406 are described in greater detail below.

FIG. 5 illustrates an assembly view of the optic mounting system 220, including cover plate 326, which is shown in phantom. In some embodiments, the cover plate 326 of the optic mounting system 220 is configured to slide onto the optic mounting plate 302. In such embodiments, the cover plate 326 may include one or more contact blocks 502. Each contact block 502 may protrude outward from the cover plate 326 and be sized to interface with, and slide along, the guide channels 406 of the optic mounting plate 302. Accordingly, the contact blocks 502 may have a thickness approximately equivalent to a depth of the guide channels 406 of the optic mounting plate 302. As further shown, the guide channels 406 and the contact blocks 502 both include guide surfaces 504. Such guide surfaces 504 are formed in the guide channels 406 and on the contact blocks 502 to be parallel to one another and allow the contact blocks 502 to slide along the guide channels 406 in a directionally controlled manner to apply a biasing force in a direction non-normal to the periphery of the optical element 314. In some embodiments, the guide surfaces 504 may be lubricated, treated, or otherwise comprise a material or structure suited for the relative sliding of the contact blocks 502 within the guide channels 406.

As the contact blocks 502 slide along the guide channels 406 toward the optical elements 314 in a diagonal direction, the contact blocks 502 contact the biasing elements 324. The contact blocks 502 may include contact surfaces 506 formed in the contact blocks 502 to contact the biasing element 324. In some embodiments, the contact surfaces 506 are v-shaped to contact and retain the biasing elements 324 relative to the contact blocks 502. The contact surface 506 may be rounded to match a geometry of the biasing element 324 or may be angular or otherwise differ from a geometry of the biasing element 324. In some embodiments, the contact surface 506 is configured to match a geometry of the biasing element 324 when the biasing element 324 is fully compressed to maximize stability of the biasing element 324 and, in turn, the optical element 314.

As described above, the cover plate 326 may be configured to slide or translate into position relative to the optic mounting plate 302 after pressing the cover plate 326 against the optic mounting plate 302 by moving the contact blocks 502 through the guide channels 406. The sliding of the cover plate 326 may be in a relatively angular direction 510. In response to sliding of the cover plate 326 relative to the optic mounting plate 302, the cover plate 326 engages the contact blocks 502 to the biasing element 324 to apply force to the biasing elements 324 at the contact surface 506 of the contact blocks 502. The force applied to the biasing elements 324 causes the biasing element 324 to press against the optical element 314. The biasing element 324 may elastically deform and extend outward in vertical and horizontal directions 508 in response to forces between the contact blocks 502 and the optical element 314, as shown in FIG. 5. In turn, the biasing element applies to the optical element 314 to press the optical element 314 into place against the registration surfaces 316.

By pressing the optical element 314 against the registration surfaces 316, the optical element 314 is properly aligned for laser transmission. The optical element 314 may be a diffractive optical element (DOE) or the like to form a pattern or otherwise manipulate the laser light based on a surgical procedure or other desired effect.

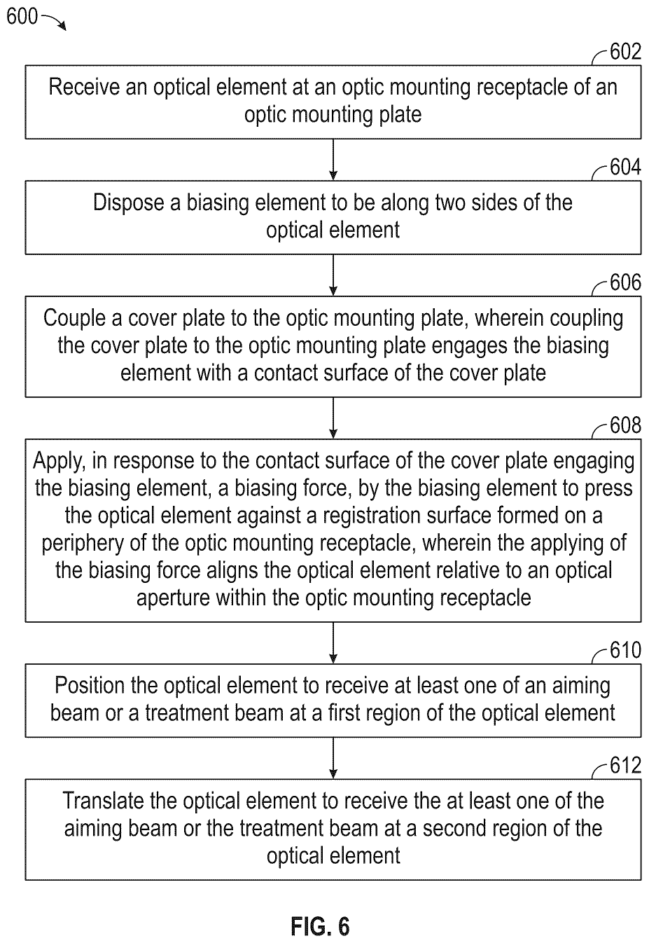

FIG. 6 illustrates a method 600 of assembling and using the optic mounting system of FIG. 3 in a surgical laser system. At block 602, an optical element (e.g., the optical element 314 of FIG. 3) is placed in an optic mounting receptacle (e.g., the optic mounting receptacle 310 of FIG. 3) of an optic mounting plate (e.g., the optic mounting plate 302 of FIG. 3) of the optic mounting system. The optic mounting plate may already be positioned and aligned with other optical relay devices of an optical relay system of the surgical laser system.

At block 604, a biasing element (the biasing element 324 of FIG. 3) is placed against at least two sides of the optical element. In some embodiments, the biasing element may be disposed in a biasing recess (e.g., the biasing recess 322 of FIG. 3) coupled to the optic mounting receptacle and having a geometry to facilitate movement of the biasing element within the biasing recess.

At block 606, a cover plate (e.g., the cover plate 326 of FIG. 3) is coupled to the optic mounting plate. Coupling the cover plate to the optic mounting plate may include pressing the cover plate to the optic mounting plate, and then sliding or translating the cover plate (while pressed against the optic mounting plate) to engage the biasing element against a contact surface (e.g., the contact surface 506 of FIG. 5) of the cover plate.

At block 608, in response to the contact surface of the cover plate engaging the biasing element, the biasing element applies a biasing force against the optical element. The biasing force may be applied in a relatively angular direction (e.g., the angular direction 510 of FIG. 5) such that the force elastically deforms the biasing element and causes the biasing element to apply force to press the optical element against a registration surface (e.g., the registration surface 316 of FIG. 3) formed on a periphery of the optic mounting receptacle. Applying the biasing force with the biasing element against the optical element may align the optical element relative to an optical aperture (e.g., the optical aperture 312 of FIG. 3) within the optic mounting receptacle. By positioning the optical element within the optic mounting receptacle, the optical element may be at least partially aligned with respect to the optical aperture and allow for proper alignment of the optical element with other optical relay devices in the surgical laser system, which enables precise laser beam transmission through a desired region of the optical element and, and, in some embodiments, facilitates precise optical manipulation of the laser beam(s) by the optical element.

At block 610, the optical element may be disposed in a first position to receive at least one of an aiming beam and/or a treatment beam at a first region of the optical element.

At block 612, the optic mounting plate of the optic mounting system may be translated to dispose the optical element in a second position to receive the at least one of the aiming beam or the treatment beam at a second region of the optical element.

As described above, the optic mounting system may include one or more optical elements. In an example of the optic mounting system having a single optical element, the optical element may be positioned to direct an aiming beam to a treatment site and similarly direct a treatment beam to the treatment site. Each of the aiming beam and the treatment beam may be passed through a first region of the optical element to apply a first effect to the aiming and treatment beams. The optic mounting system may be translated to then pass the aiming and treatment beams through a second region of the optical element to apply a second effect to the aiming and treatment beams that is different from the first effect. The first effect and the second effect may, for example, form different patterns, beam widths, or the like.

In an example of the optic mounting system having multiple optical elements, an aiming optical element may be positioned in the optic mounting system to receive the aiming beam and a treatment optical element may be positioned in the optic mounting system to receive the treatment beam. Each of the aiming optical element and the treatment optical element may have corresponding first and second regions formed therein. Translation of the optic mounting system results in a shift of the aiming and treatment beams from the first region of each optical element to a second region.

Accordingly, the embodiments described herein allow for using an optic mounting system to align an optical element for more accurate transmission of laser light. The assembly of the optic mounting system is repeatable with improved consistency in alignment of the optical element. The arrangement of the optic mounting system is resilient to high-speed movement without misalignment of the optical element or relative displacement of other components of the optic mounting system.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A system for laser alignment, the system comprising:
an optic mounting plate comprising:
an optic mounting receptacle formed on a first side of the optic mounting plate, the optic mounting receptacle configured to accept an optical element, the optic mounting receptacle comprising:
a registration surface formed on a periphery of the optic mounting receptacle, the registration surface facing inward on the optic mounting receptacle;
an optical aperture disposed within the optic mounting receptacle, the optical aperture extending through a thickness of the optic mounting plate from the first side of the optic mounting plate to a second side of the optic mounting plate; and
a biasing recess formed in the optic mounting plate to extend along two sides of the optic mounting receptacle;
a biasing element disposed in the biasing recess and shaped to extend along the two sides of the optic mounting receptacle to apply a biasing force to the optical element to press the optical element against the registration surface to align the optical element relative to the optic mounting plate; and
a cover plate configured to couple to the first side of the optic mounting plate to cover the biasing recess, wherein the cover plate is configured to:
engage the biasing element with a contact surface of the cover plate; and
load the biasing element to apply the biasing force to the optical element.

2. The system of claim 1, wherein the biasing element is a spring.

3. The system of claim 1, wherein the optical element is a diffractive optical element.

4. The system of claim 1, wherein the optic mounting plate comprises a guide surface to align the cover plate relative to the optic mounting plate.

5. The system of claim 1, wherein:
the optic mounting receptacle comprises a first optic mounting receptacle and a second optic mounting receptacle; and
the optical aperture comprises a first optical aperture and a second optical aperture, the first optical aperture corresponding to the first optic mounting receptacle and the second optical aperture corresponding to the second optic mounting receptacle.

6. The system of claim 5, wherein the optic mounting plate further comprises a pass-through disposed in the optic mounting plate between the first optic mounting receptacle and the second optic mounting receptacle.

7. A method for laser alignment, the method comprising:
receiving an optical element at an optic mounting receptacle of an optic mounting plate;
disposing a biasing element to be along two sides of the optical element;
coupling a cover plate to the optic mounting plate, wherein coupling the cover plate to the optic mounting plate engages the biasing element with a contact surface of the cover plate; and
in response to the contact surface of the cover plate engaging the biasing element, applying, by the biasing element, a biasing force to press the optical element against a registration surface formed on a periphery of the optic mounting receptacle, wherein the applying of the biasing force aligns the optical element relative to an optical aperture within the optic mounting receptacle.

8. The method of claim 7, wherein applying the biasing force by the biasing element comprises elastically deforming the biasing element.

9. The method of claim 7, wherein coupling the cover plate to the optic mounting plate comprises translating the cover plate along a guide surface of the optic mounting plate.

10. The method of claim 9, wherein the translating of the cover plate is in a same direction as the applying of the biasing force by the biasing element to press the optical element against the registration surface.

11. The method of claim 7, wherein coupling the cover plate to the optic mounting plate comprises positioning a coupling element to extend through the cover plate and interface with the optic mounting plate.
